(19) United States
(12) Patent Application Publication
(10) Pub. No.: US 2006/0008916 A1
(43) Pub. Date: Jan. 12, 2006

(54) Method for measuring strontium isotope ratios using mass spectrometer

(76) Inventors: Guang-sheng Qiao, Beijing (CN); Zhu-yin Chu, Beijing (CN)

Correspondence Address: PRO-TECTOR INTERNATIONAL SERVICES, 20775 NORADA CT., SARATOGA, CA, 95070, US

(21) Appl. No.: 11/171,611

(22) Filed: Jun. 30, 2005

(30) Foreign Application Priority Data
Jun. 30, 2004 (CN) 200410062205.9

(51) Int. Cl. G01N 33/20

(52) U.S. Cl. 436/79; 436/173

(57) ABSTRACT

The invention put forward an improved method for isotope dilution analysis of strontium using mass spectrometer. The sample of strontium is measured by a multi-collector mass spectrometer which equipped with no less than five collectors. The isotopes of strontium are statically collected by the collectors: ion beams with different mass numbers will be collected by different collectors. The voltages of the outputs of the collectors are read and amplified by a dynamic virtual amplifier. Then equations which include some variables and the output voltages of every amplifiers are established. The solutions of the equations will be the isotope ratios of the sample being measured. The invention can combine two analyses, one for isotope ratio and the other for concentration of strontium, together into one so that the measurement procedure is simplified.

### FIELD OF THE INVENTION

The invention relates to strontium isotope mass spectrometry and isotope geochronology.

### BACKGROUND AND PRIOR ART

Strontium has four naturally occurring isotopes, ⁸⁴Sr, ⁸⁶Sr, ⁸⁷Sr and ⁸⁸Sr all of them are stable. Their isotope abundances are approximately 0.56 percent, 9.86 percent, 7.02 percent and 82.56 percent respectively. The isotopic abundances of strontium are variable because of the formation of the radiogenic ⁸⁷Sr by the β⁻ decay of naturally occurring ⁸⁷Rb. For this reason precise isotopic composition of strontium in a rock or mineral that contains rubidium depends on the age and Rb/Sr ratio of that rock or mineral. Generally the ratio ⁸⁷Sr/⁸⁶Sr is used to express relative abundance of strontium in geochronology. Accurately measuring the ratio ⁸⁷Sr/⁸⁶Sr and the concentration of strontium in a rock or mineral can be used to determine the age of that rock or mineral. Because of the concentration of strontium in geology samples is very low, the isotope ratio and concentration of strontium are determined by undiluted and diluted isotope analysis both of them include chemical analysis and mass spectrometric analysis. The method of isotope dilution analysis of strontium has been existed. It based on the determination of isotope composition of strontium in a mixture of a known quantity of “spike” with unknown quantity of sample strontium. The spike of strontium is a solution contains known concentration and known isotope composition of which ⁸⁴S is enriched and here ⁸⁴Sr/⁸⁶Sr═S1, ⁸⁸Sr/⁸⁶S═S2 and ⁸⁷Sr/⁸⁶S═Si. Two of the naturally occurring isotope ratios in a sample are known as constants ⁸⁴Sr/⁸⁶Sr═N1 and ⁸⁷Sr/⁸⁶Sr═N2 where N1=0.056 584and N2=8.37521,

There are two ways to determine the isotope ratios and the concentration of strontium in a rock or mineral:
1. A conventional way of dating a rock or mineral with a multi-collector mass spectrometric isotope dilution technique involves two procedures: an undiluted run, by which the naturally occurring isotope ratio ⁸⁷Sr/⁸⁶Sr of the sample can be measured; a diluted run, by which the concentration of strontium of the sample can be determined. The two procedures are almost the same: both of them involve one chemical analysis and one mass spectrometric isotope analysis. For example the new product of Finnigan Mat company equipped with virtual amplifier which based on the patent U.S. Pat. No. 342,152 measures undiluted isotopes of strontium of a sample with a high precision, but in order to determine the concentration of strontium of the sample another run of a diluted analysis have to be done.
2. A simple way of dating a rock or mineral with multi-collector mass spectrometric isotope dilution technique in which the diluted run and undiluted run are combined together into one as suggested by the inventor in the patent No. ZL97100609 in Chinese. The prior invention is created for measuring both diluted and undiluted isotope analysis of strontium using multi-collector mass spectrometer. The advantages are as follows: it can combine diluted run and undiluted run together into one, so that the measuring procedure can be only one chemical analysis and one mass spectrometric analysis; it perfectly solves the difference of gains and corrects isotope fractionation automatically.

Both of the two ways still have some disadvantages.

In the first way above, as an example, although the new product of Finnigan Mat company equipped with virtual amplifier which based on the patent U.S. Pat. No. 342,152 solves the problem of “gain calibration” with a “novel method”, but it still has some disadvantages: the gain calibration must be done after at least extra j times virtual jumps between blocks of measurement, where j is the number of channels of the collectors; it can not normalize diluted isotope analysis and therefore for geochronology sample of Sr two procedures must be done: a undiluted run, by which the naturally occurring isotope ratio ⁸⁷Sr/⁸⁶Sr of the sample can be measured; a diluted run, by which the concentration of strontium of the sample can be measured.

The second way mentioned above is based on the invention of patent No. ZL97100609N which is suited to a general multi-collector thermal mass spectrometer equipped without virtual amplifier. The peak jumping is done dynamically through changes of magnet field. It also has some disadvantages:

1, the field strength changing quickly from one channel to another need a response time and at the same time the reproducibility of the position of peaks is not exactly the same as the peaks jumping backward and forward.

2, as the field strength changes, tails of the moving peaks will interfere with the background of the amplifiers.

In order to get good result, following every time of peak jumping there must be a waiting time about 2 or 4 seconds, therefore the measuring time is longer.

### SUMMARY OF THE INVENTION

The object of the invention is to improve the isotope analysis of strontium. A novel method for isotope analysis of strontium has been invented here. The method can combine diluted analysis with undiluted analysis of strontium together into one run which makes the analysis procedure of strontium more simplified.

In order to reach the object, an improved method for isotope analysis of strontium is created. The isotopes of the mixed sample of normal strontium with spike will be measured by a multi-collector mass spectrometer. The method includes □

A mass spectrometer equipped with no less than five collectors Fj (j=1, 2, . . . , 5) which statically collect the ion beams of mass numbers of the isotopes of strontium, i.e. the ion beams of mass number 84, 85, 86, 87 and 88 are statically collected by the collects F1, F2, F3, F4 and F5 respectively;

After no less than three times measuring by virtual peak jumping, the readings of different collectors will be as follows respectively: in the first measuring the output voltages of A1, A2, A3, A4 and A5 are v1j(j=1, 2, . . . , 5); in the second the output voltages of A1, A2, A3, and A4 are v2j(j=1, 2, . . . , ); and in the third the output voltages of A1, A2 and A3 are v3j (j=1, 2,3)

Solving simultaneous equations by combining one of the following no less than three equations:
□⁸⁴Sr/⁸⁶Sr_{□n}·□⁸⁸Sr/⁸⁶Sr_{□n}□□v₁₁·v_{33□}/□v₁₃·v_{31□}
[□⁸⁷Sr/⁸⁶Sr_{□n}·□⁸⁷Sr/⁸⁸Sr_{□n}]₁□□v₁₄·v_{23□}/□v₁₃·v_{24□}
[□⁸⁷Sr/⁸⁶Sr_{□n}·□⁸⁷Sr/⁸⁸Sr_{□n}]₂□□v₂₃·v_{32□}/□v₂₂·v_{33□}
with one of the formulas:
for undiluted analysis:
⁸⁸Sr/⁸⁶Sr_{□n=8.37521}
for diluted analysis:
[S₁—□⁸⁴Sr/⁸⁶Sr_{□n}]/[□⁸⁴Sr/⁸⁶Sr_{□n}—N₁]□[S₂—□⁸⁸Sr/⁸⁶Sr_{□n}]/[□⁸⁸Sr/⁸⁶Sr_{□n}—N₂]
where (⁸⁴Sr/⁸⁶Sr_{□n},□⁸⁷Sr/⁸⁶Sr_{□n} and □⁸⁸Sr/⁸⁶Sr_{□n} are normalized mixed ratios and they are variables of the equations; S1, Si and S2 denote the known isotope ratios ⁸⁴Sr/⁸⁶Sr□⁸⁷Sr/⁸⁶Sr and ⁸⁸Sr/⁸⁶Sr of the spike respectively, N1 and N2 denote naturally occurring ratios ⁸⁴Sr/⁸⁶Sr and ⁸⁸Sr/⁸⁶Sr of strontium.
the solutions of the simultaneous equations will be:
[□⁸⁷Sr/⁸⁶Sr_{□n}]₁□\sqrt{(V₁₄·V₂₃)·K/(V₁₃·V₂₄)}
[□⁸⁷Sr/⁸⁶Sr_{□n}]₂□\sqrt{(V₂₃·V₃₂)·K/(V₂₂·V₃₃)}
where K is the value of □⁸⁸Sr/⁸⁶Sr_{□n.}
Two values of □⁸⁷Sr/⁸⁶Sr_{□n} can be obtained in one cycle of scan. The values of □⁸⁴Sr/⁸⁶Sr_{□n} and □⁸⁸Sr/⁸⁶Sr_{□n} can also be solved by solving the simultaneous equations.

The following formula can be used to correct the contribution of spike to the ratio □⁸⁷Sr/⁸⁶Sr_{□n:}
Nᵢ□□⁸⁷Sr/⁸⁶Sr_{□n}+[□⁸⁷Sr/⁸⁶Sr_{□n}−Sᵢ]·□N₁−□⁸⁴Sr/⁸⁶Sr_{□n}□/□□⁸⁴Sr/⁸⁶Sr_{□n}−S₁□
where Ni denotes the isotope ratio □⁸⁷Sr/⁸⁶Sr_{□n} of the sample to which the contribution of the spike has been corrected).

If the quantity of spike is zero then □⁸⁴Sr/⁸⁶Sr_{□n->N1}, the second term in correct formula will be zero and the formula still be valid, this kind of zero spike dilution analysis will be called “virtual dilution analysis”.

The invention created a method which includes: measuring the output voltage using virtual amplifier; building up equations based on the measured results of every collectors and on the unknown ratios; solving the equations and getting the isotope ratios of the sample. The invented method has many benefits: 1, it is independent of gains of different amplifiers; 2, it eliminates isotope fractionation automatically; and 3, it can correct the contribution of spike to the natural occurring ratio ⁸⁷Sr/⁸⁶Sr of strontium. So that the diluted run and the undiluted run can be combined together into one. All of the benefits make the procedure of measurement simplified and easier to operate and the measured result reliable;

in order to make the examiner deeply understand for reaching the object of the invention, some useful examples for implication and their schematic diagrams are listed and show what techniques are adopt and what effects will be get

### BRIEF DESCRIPTION OF THE DRAWINGS

FIG. 1 is schematic diagram of an example for the first measurement of “peak jumping” by the virtual amplifier mounted on a thermal mass spectrometer;

FIG. 2 is schematic diagram of example 1 for the second measurement of “peak jumping” by the virtual amplifier mounted on a thermal mass spectrometer;

FIG. 3 is schematic diagram of example 1 for the third measurement of “peak jumping” by the virtual amplifier mounted on a thermal mass spectrometer;

FIG. 4 is schematic diagram of example 1 for the fourth measurement of “peak jumping” by the virtual amplifier mounted on a thermal mass spectrometer;

FIG. 5 is schematic diagram of example 1 for the fifth measurement of “peak jumping” by the virtual amplifier mounted on a thermal mass spectrometer;

FIG. 6 is schematic diagram of example 2 for the second measurement of “peak jumping” by the virtual amplifier mounted on a thermal mass spectrometer;

FIG. 7 is schematic diagram of example 2 for the third measurement of “peak jumping” by the virtual amplifier mounted on a thermal mass spectrometer;

### DETAILED DESCRIPTION OF THE PREFERRED EMBODIMENT

The following examples describe the practices of the invention in detail but they are not used to limit the invention.

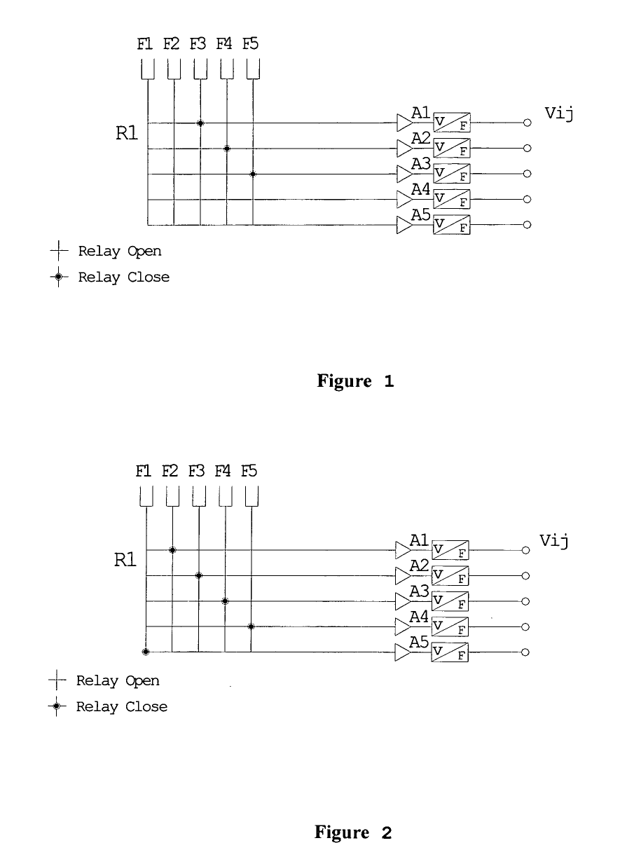

The invention can be implicated to a thermal mass spectrometer equipped with virtual amplifier. For measuring dilution isotopes of strontium a new Finnigam Mat thermal mass spectrometer, Triton T1, is selected in the examples. The virtual amplifier based on the invention of U.S. Pat. No. 342,152 and mounted on the mass spectrometer Triton T1 includes nine cups F1, F2 . . . F9 and nine amplifiers A1,A2 . . . A9. The gains of the amplifiers are G(j) (j=1, 2, . . . 9). In the examples of the invention only five cups Fj(j=1, 2, . . . , 5) and five amplifiers Aj (J=1, 2, . . . , 5) are used as shown in FIG. 1. The important point is that the ion beams of the isotopes of strontium should keep stable and being collected by five cups statically and correspondingly. In order to complete one cycle of scan and read out the signals of isotope of strontium from the cups in every integrating time ti, a quintuple amplifier is connected to a quintuple cup dynamically and respectively by a relay matrix R1 (a virtual amplifier is composed in this way). The ratios and concentration of strontium can be obtained in every cycle of scan. The operating is detailed as follows:

Adjusting the magnet field and the collectors so that the ion beams of ⁸⁴Sr, ⁽⁸⁵Rb) ⁸⁶Sr, ⁸⁷Sr and ⁸⁸Sr of diluted or undiluted sample of strontium are collected statically by the collectors Fj (j=1, 2, . . . , 5) through peak centering technique and supposing that the amplified ion beams are ⁸⁴I, ⁸⁵I ⁸⁶I, ⁸⁷I and ⁸⁸I respectively. As shown in FIG. 1, a virtual amplifier is composed of a quintuple amplifier Aj (j=1, 2, . . . , 5) which is connected to a quintuple cup Fj(j=1, 2, . . . , 5) through a relay matrix R1. The controlling of the relay matrix by the software of the mass spectrometer is not belong to the invention and should not be described here.

### EXAMPLE 1

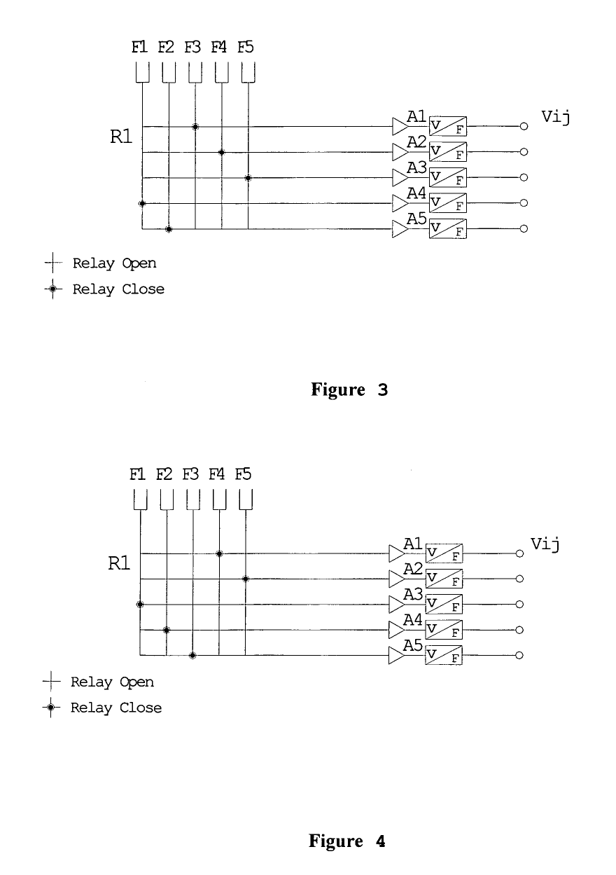

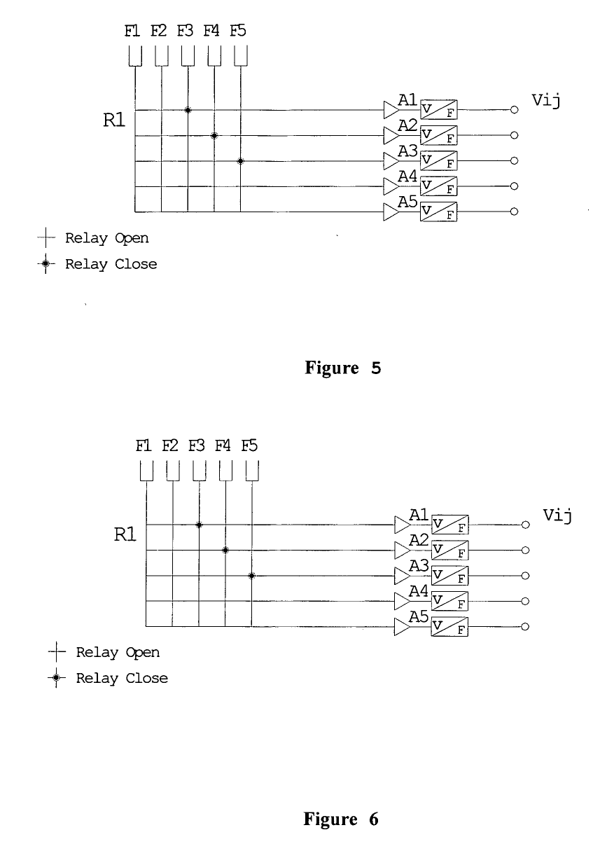

As shown in FIG. 1 to FIG. 5, there are five integrating times ti (i=1, 2, . . . , 5) in one cycle of scan (although in the invention, the integrating times can be three as ti (i=1, t2, t3). In order to describe easily, we suppose that the outputs of amplifiers A1, A2, A3, A4 and A5 are vij. and the gains of them are G(j) (j=1, 2, . . . , 5) respectively.

The figures from FIG. 1 to FIG. 5 show the “peak jumping” schematic diagrams of the first example for practicing the invention. The figures include five times integrating t1 to t5 which just compose a procedure of a full rotate cycle of scan of a thermal ionization mass spectrometer equipped with virtual amplifier. Under the controlling of the rely matrix, the outputs of the amplifiers G(j) are corresponding to the signals of ⁸⁴Sr, ⁵Rb, ⁸⁶Sr, ⁸⁷Sr and ⁸⁸Sr in turn in a full rotated scan. The integrated results can be filled in the following table which is a spectrum table of one full rotated peak jumping cycle of scan.

Spectrum Table
of One Quintuple Collector of 5-Virtual Peak Jumping Scan

| | V₁ | V₂ | V₃ | V₄ | V₅ |
|---|---|---|---|---|---|
| t1 | 84 | 85 | 86 | 87 | 88 |
| t2 | 85 | 86 | 87 | 88 | 84 |
| t3 | 86 | 87 | 88 | 84 | 85 |
| t4 | 87 | 88 | 84 | 85 | 86 |
| t5 | 88 | 84 | 85 | 86 | 87 |

V₁, V,₃ . . . V₅ are the outputs of amplifiers A₁, A₂, . . . A₅ respectively
t₁, t₂ . . . t₅ are integrated times of every peak jumping from t1 to t5 respectively.
The numbers in the table represent the intensities of integrated signals corresponding to the mass numbers.

The table can be expressed as a matrix [vij]:

$$\begin{matrix}Spectrum Matrix \\ \vert \begin{matrix}{v}_{11} & {v}_{12} & {v}_{13} & {v}_{14} & {v}_{15} \\ {v}_{21} & {v}_{22} & {v}_{23} & {v}_{24} & {v}_{25} \\ {v}_{31} & {v}_{32} & {v}_{33} & {v}_{34} & {v}_{35} \\ {v}_{41} & {v}_{42} & {v}_{43} & {v}_{44} & {v}_{45} \\ {v}_{51} & {v}_{52} & {v}_{53} & {v}_{54} & {v}_{55}\end{matrix} \vert\end{matrix}$$

If vᵢⱼ presents one element of the matrix [vij] then
v₁₁=⁸⁴I·G□1□·f□t₁□□
v₂₁=⁸⁵I·G□1□·f□t₂□□
v₃₁=⁸⁶I·G□1□·f□t₃□□
v₄₁=⁸⁷I·G□1□·f□t₄□□
v₅₁=⁸⁸I·G□1□·f□t₅□□
v₁₂=⁸⁵I·G□2□·f□t₁□□
v₂₂=⁸⁶I·G□2□·f□t₂□□
v₃₂=⁸⁷I·G□2□·f□t₃□□
v₄₂=⁸⁸I·G□2□·f□t₄□□
v₅₂=⁸⁴I·G□2□·f□t₅□□
v₁₃=⁸⁶I·G□3□·f□t₁□□
v₂₃=⁸⁷I·G□3□·f□t₂□□
v₃₃=⁸⁸I·G□3□·f□t₃□□
v₄₃=⁸⁴I·G□3□·f□t₄□□
v₅₃=⁸⁵I·G□3□·f□t₅□□
v₁₄=⁸⁷I·G□4□·f□t₁□□
v₂₄=⁸⁸I·G□4□·f□t₂□□
v₃₄=⁸⁴I·G□4□·f□t₃□□
v₄₄=⁸⁵I·G□4□·f□t₄□□
v₅₄=⁸⁶I·G□4□·f□t₅□□
v₁₅=⁸⁸I·G□5□·f□t₁□□
v₂₅=⁸⁴I·G□5□·f□t₂□□
v₃₅=⁸⁵I·G□5□·f□t₃□□
v₄₅=⁸⁶I·G□5□·f□t₄□□
v₅₅=⁸⁷I·G□5□·f□t₅□□
in which
f□t₁□□f□t₂□□f□t₃□□f□t₄□and f□t₅□ are the fluctuating factors of the total ion beam during different integrating time t₁□t₂□t₃□t₄□t₅, and G(j) denote the gains of the amplifiers A1, A2 . . . , A5, respectively. According to some regular, selecting elements vᵢⱼ and eliminating the same factors, an equation can be established:
□v₁₁·v_{33□}/□v₁₃·v_{31□}□□⁸⁴I·⁸⁸I_{□}/□⁸⁶I·⁸⁶I_{□}□□⁸⁴Sr/⁸⁶Sr_{□m}·□⁸⁸Sr/⁸⁶Sr_{□m} (1)
where the index m denotes measured value.

By the mass fractionation correcting equation in the first reference paper ( G. J. Wasserburg et al., Precise determination of Sm/Nd ratio, Sm and Nd abundances in standard solutions, page 2315), if

$$• {}^{88} Sr / {}_{}^{86}{Sr}{}_{• n}^{} • • {}^{88} Sr / {}_{}^{86}{Sr}{}_{• m}^{} \cdot • 1 + α {•}^{( 88 - 86 )} = • {}^{88} Sr / {}_{}^{86}{Sr}{}_{• m}^{} \cdot • 1 + α {•}^{2} \qquad ( 2 )$$

then
□⁸⁴Sr/⁸⁶Sr_{□n}□□⁸⁴Sr/⁸⁶Sr_{□m}·□1+α□⁽⁸⁴⁻⁸⁶⁾□□⁸⁴Sr/⁸⁶Sr_{□m}·□1+α□⁻² (3)
whereαis isotope fractionation factor per atomic mass unit and the index n denotes normalized ratio.
Substituting (2) and (3) into (1), we get
[□⁸⁴Sr/⁸⁶Sr_{□n}·□⁸⁸Sr/⁸⁶Sr_{□n}]₁□□v₁₁·v_{33□}/□v₁₃·v_{31□} (4)
where □⁸⁴Sr/⁸⁶Sr_{□n} and □⁸⁸Sr/⁸⁶Sr_{□n} are just the normalized dilution isotope ratios we want to get.
Using similar procedure we can establish more similar equations from the spectrum matrix [vᵢⱼ]:
[□⁸⁷Sr/⁸⁶Sr_{□n}·□⁸⁷Sr/⁸⁸Sr_{□n}]₁□□v₁₄·v_{23□}/□v₁₃·v_{24□} (5)
[□⁸⁷Sr/⁸⁶Sr_{□n}·□⁸⁷Sr/⁸⁸Sr_{□n}]₂□□v₂₃·v_{32□}/□v₂₂·v_{33□} (6)
[□⁸⁷Sr/⁸⁶Sr_{□n}·□⁸⁷Sr/⁸⁸Sr_{□n}]₃□□v₃₂·v_{41□}/□v₃₁·v_{42□} (7)
[□⁸⁷Sr/⁸⁶Sr_{□n}·□⁸⁷Sr/⁸⁸Sr_{□n}]₄□□v₄₁·v_{55□}/□v₄₅·v_{51□} (8)
[□⁸⁴Sr/⁸⁶Sr_{□n}·□⁸⁸Sr/⁸⁶Sr_{□n}]₂□□v₃₄·v_{51□}/□v₃₁·v_{54□} (9)
where all the ratios of strontium are just the normalized dilution isotope ratios which we want to get.
From the equations□4□, (5), (6), (7), (8) and □9□□we can draw a interesting conclusion:
Using the invented technology, we can establish some equations every one of which contains products of pairs of normalized ratio of Sr. The product can be calculated through selecting elements vᵢⱼ of a spectrum matrix [vᵢⱼ] according to some regulation. The equations are independent of gains of the amplifiers and also independent of isotope fractionation factor α. Solving the equations the normalized ratios of Sr can be obtained directly.
For undiluted analysis, □⁸⁸Sr/⁸⁶Sr_{□n} □8.37521, the equations can be easily solved.
For dilution analysis, the equations can neither be solved lonely nor be solved by combining each other. Firstly, □⁸⁴Sr/⁸⁶Sr_{58 n}and □⁸⁸Sr/⁸⁶Sr_{□n} can be solved using simultaneous equations of the combination of equation (4) or (5) with the following formulae which quoted from the second reference paper (Boelrijk, N. A. I. M., “A general formula for “double isotope dilution analysis”, Chemical Geology, 3(1968), 323-325):
[S₁−□⁸⁴Sr/⁸⁶Sr_{□n}]/[□⁸⁴Sr/⁸⁶Sr_{□n}−N₁]□[S₂−□⁸⁸Sr/⁸⁶Sr_{□n}]/[□⁸⁸Sr/⁸⁶Sr_{□n}−N₂] (10)
where S1, and S2 represent isotope ratios ⁸⁴Sr/⁸⁶Sr□ and ⁸⁸Sr/⁸⁶Sr of the spike; N1 and N2 are constants and represent naturally occurring isotope ratios ⁸⁴Sr/⁸⁶Sr and ⁸⁸Sr/⁸⁶Sr respectively.
Two pairs of solutions of the ratios ⁸⁴Sr/⁸⁶Sr□ and ⁸⁸Sr/⁸⁶Sr can be solved and the others may be solved easily.
Supposing one of the solutions of ratio□⁸⁸Sr/⁸⁶Sr_{□n} is
[□⁸⁸Sr/⁸⁶Sr_{□n}]₂□K (11)
substitute equation (11) into equation (5), (6), (7) and (8) respectively the solutions of ratio□⁸⁷Sr/⁸⁶Sr_{□n} will be
[□⁸⁷Sr/⁸⁶Sr_{□n}]₁□\sqrt{(V₁₄·V₂₃)·K/(V₁₃·V₂₄)} (12)
[□⁸⁷Sr/⁸⁶Sr_{□n}]₂□\sqrt{(V₂₃·V₃₂)·K/(V₂₂·V₃₃)} (13)
[□⁸⁷Sr/⁸⁶Sr_{□n}]₃□\sqrt{(V₃₂·V₄₁)·K/(V₃₁·V₄₂)} (14)
[□⁸⁷Sr/⁸⁶Sr_{□n}]₄□\sqrt{(V₄₁·V₅₅)·K/(V₅₁·V₄₅)} (15)
Therefore, four values of ratios □⁸⁷Sr/⁸⁶Sr_{□n} and two values of ratios □⁸⁴Sr/⁸⁶Sr_{□n} are obtained in one cycle of scan. This character cannot only improve the efficacy of analysis but can also monitor the measuring system. Normally the values of the same ratio should be exactly the same respectively within the fluctuation of the measuring system.

The contribution of spike to naturally occurring isotope ratio (⁸⁷Sr/⁸⁶Sr) can be corrected by the following formula which is similar with the equation (3.12) quoted from reference 3 (Derek york: The Earth's Age and Geochronology) or the equation (2) quoted from reference 4 (Qiao G-sh, Normalization of isotope dilution analysis—A new program for isotope mass spectrometric analysis, Sciences in China (Series A), No. 10 (1988), page 1264)
Nᵢ□□⁸⁷Sr/⁸⁶Sr_{□n}+[□⁸⁷Sr/⁸⁶Sr_{□n}−Sᵢ]·□N₁−□⁸⁴Sr/⁸⁶Sr_{□n}□/□□⁸⁴Sr/⁸⁶Sr_{□n}−S₁□□ (16)
where Ni is the isotope ratio ⁸⁷Sr/⁸⁶Sr and has been normalized and corrected the contribution of spike, i.e., Ni is just the naturally occurring isotope ratio ⁸⁷Sr/⁸⁶Sr of the sample.

If the quantity of spike is zero then □⁸⁴Sr/⁸⁶Sr_{□n->N1}, the second term in the correct formula will be zero and the formula still be valid. This kind of zero spike dilution analysis will be called “virtual dilution analysis” which had been suggested by the inventor in reference 4.

The concept of “virtual diluted isotope analysis” is a useful suggestion. It makes that both the dilution analysis and the undiluted analysis can be done by the same mass spectrometric method and the same program, therefore the analysis procedure is simplified and the quality of the measure result is improved.

### EXAMPLE 2

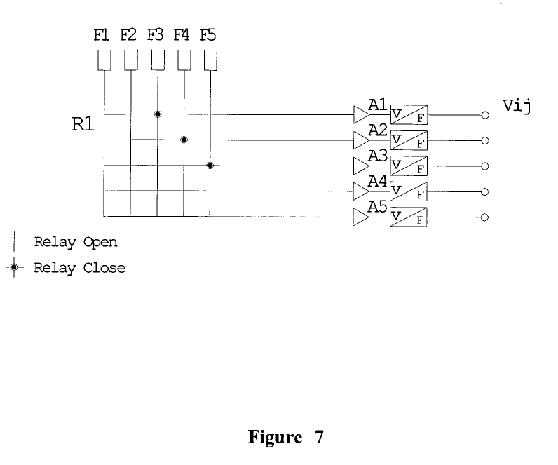

Basically the example 2 is similar to example 1. The difference is as follows: the integrating times ti is three ( i=1, 2, 3) instead of five (i=1, 2, . . . , 5) as example 1 where the scan is a full rotated peak jumping cycle; sometimes all the output signals of the A1, A2, A3, A4 and A5 are read but sometimes only part of them are read as shown in FIG. 1, FIG. 6 and FIG. 7.

For diluted isotope analysis, the first work is chemical analysis which includes isotope dilution procedure. The isotope ratios of the spike are known as ⁸⁴Sr/⁸⁶Sr═S1□⁸⁸Sr/⁸⁶Sr═S2 and ⁸⁷Sr/⁸⁶Sr═Si. In the sample two of the naturally occurring isotope ratios ⁸⁴Sr/⁸⁶Sr═N1 and ⁸⁸Sr/⁸⁶Sr═N2 where N1 and N2 are constants 0.056584 and 8.37521 respectively.

The second is isotope dilution analysis using thermal ionization mass spectrometer. After the ion beam appeared in the monitor of the mass spectrometer, adjust the magnet field and the collectors so that the ion beams of ⁸⁴Sr, ⁸⁵Rb ⁸⁶Sr, ⁸⁷Sr and ⁸⁸Sr of the sample are collected stably and statically by the collectors Fj (j=1, 2, . . . , 5) through peak centering technique. Suppose that the currents of the amplified ion beams are ⁸⁴I, ⁸⁵I^{,86}I, ⁸⁷I and ⁸⁸I respectively. Then:

1, In the first integrating time t1 as shown in FIG. 1, which is the first “peak jumping” schematic diagram of thermal mass spectrometer equipped with a virtual amplifier, the amplifiers Aj(j=1, 2, . . . , 5) read the signals of isotopes ⁸⁴Sr, ⁸⁵Rb ⁸⁶Sr, ⁸⁷Sr and ⁸⁸Sr respectively controlled by the relay matrix R1.

2, In the second integrating time t2 as shown in FIG. 6, which is the second “peak jumping” schematic diagram of thermal mass spectrometer equipped with a virtual amplifier, the amplifiers Aj(j=1, 2, . . . , 4) read the isotopes ⁸⁵Rb ⁸⁶Sr, ⁸⁷Sr and ⁸⁸Sr respectively controlled by the relay matrix R1. It should be noted that the amplifier A5 is not used and should be empty.

3, In the final integrating time t3 as shown in FIG. 7, which is the third “peak jumping” schematic diagram of thermal mass spectrometer equipped with a virtual amplifier, the amplifiers Aj (j=1, 2, 3) read the isotopes ⁸⁶Sr, ⁸⁷Sr and ⁸⁸Sr respectively controlled by the relay matrix R1. It should be noted that the amplifiers A4 and A5 are not used and should be empty.

The integrated signals can be filled in the following table which is a spectrum table of one cycle of scan.

Spectrum Table of
One Quintuple Collector 3 Virtual Peak Jumping Scan

| | V1 | V2 | V3 | V4 | V5 |
|---|---|---|---|---|---|
| t1 | 84 | 85 | 86 | 87 | 88 |
| t2 | 85 | 86 | 87 | 88 | empty |
| t3 | 86 | 87 | 88 | empty | empty |

V1, V,3 . . . V5 are the outputs of amplifiers A1, A2, . . . A5 respectively
t1, t2, t3 are integrated times of the peak jumps
The numbers in the table represent the intensities of integrated signals corresponding to the mass numbers.

The spectrum table can be expressed as a matrix [vij]:

$$\vert \begin{matrix}{V}_{11} & {V}_{12} & {V}_{13} & {V}_{14} & {V}_{15} \\ {V}_{21} & {V}_{22} & {V}_{23} & {V}_{24} & {V}_{25} \\ {V}_{31} & {V}_{32} & {V}_{33} & {V}_{34} & {V}_{35}\end{matrix} \vert$$

If vᵢⱼ presents one element of the matrix [vij] then
v₁₁=⁸⁴I·G□1□·f□t₁□□
v₁₂=⁸⁵I·G□2□·f□t₁□□
v₁₃=⁸⁶I·G□3□·f□t₁□□
v₁₄=⁸⁷I·G□4□·f□t₁□□
v₁₅=⁸⁸I·G□5□·f□t₁□□
v₂₁=⁸⁵I·G□1□·f□t₂□□
v₂₂=⁸⁶I·G□2□·f□t₂□□
v₂₃=⁸⁷I·G□3□·f□t₂□□
v₂₄=⁸⁸I·G□4□·f□t₂□□
v₂₅ is empty
v₃₁=⁸⁶I·G□1□·f□t₃□□
v₃₂=⁸⁷I·G□2□·f□t₃□□
v₃₃=⁸⁸I·G□3□·f□t₃□□
v₃₄ is empty
v₃ is empty

As in example 1, selecting elements vᵢⱼ according to some regular and eliminating the same factors, equations similar to equation (1) can be established. Using the same way as in example 1, all the ratios of strontium will be solved.

Summarily, in the examples for practicing the method of the invention, there should be no less than five collectors which collect mass number 84, 85, 86, 87 and 88 respectively; and a completed cycle of scan includes no less than three times integrating which measure the signals of the mass number respectively.

Conclusion: through all of the description above, the invention has some advantages clearly as follows:

1, the gain calibration is done during measurement instead of being done before the measurement start, i.e there isn't any extra rotation of virtual peak jumping for gain calibration between every two blocks during the measurement.

2, it can normalize the isotope ratios of dilution analysis of strontium automatically, so that isotopic fractionation can be corrected and the calculated concentration of strontium will be more reliable.

3, the naturally occurring isotope ratio of strontium can be calculated from dilution analysis of strontium with the same accuracy and precise as that of undiluted analysis. Therefore the work of both chemical analysis and mass spectrometric analysis are cut down about fifty percent.

4, the invented method can correct the contamination of spike to the chemical blank or the memory of the spike inside mass spectrometer automatically.

5, in the mass spectrometric analysis, the ion beams are collected by the corresponding collectors using static scan model which overcomes the disadvantage of dynamical scan model and makes the procedure of measurement simplified and easy to operating.

6, the method can not only measure dilution analysis of strontium but it can also measure undiluted analysis, i.e., virtual dilution analysis, without any change and the precise and accuracy is better than that of the convenient method.

7, in the method, the isotope ratio and the concentration of strontium are determined from the same share of a sample; therefore the unevenness of the geology sample can be avoided.

There will be some change, revisers and/or complementary easily happened to the description based on the embodiment content. But it can be understand that all of the changes, revisers and complement are involved in the spirit and the field of the invention described above.

What is claimed is:

1. Method for measuring strontium isotope ratios using mass spectrometer which equipped with multi-collector, comprising the steps of:
the mass spectrometer equipped with no less than five collectors Fj (j=1, 2, . . . , 5) statically collect the ion beams of mass numbers of isotope of strontium, the ion beams of mass number 84, 85, 86, 87 and 88 are statically collected by the collectors A1, A2, A3, A4 and A5 respectively; after no less than three times measuring by virtual peak jumping, the readings of the collectors will be as follows respectively: in the first measuring the output voltages of A1, A2, A3, A4 and A5 are v1j(j=1, 2 . . . , 5), in the second the voltages of A1, A2, A3 and A4 are v2j (j=1, 2, . . . , ), and in the third the voltages of A1, A2 and A3 are v3j (j=1, 2,3);
Solving simultaneous equations by combination of equations:
□⁸⁴Sr/⁸⁶Sr_{□n}·□⁸⁸Sr/⁸⁶Sr_{□n}□□v₁₁·v_{33□}/□v₁₃·v_{31□}
[□⁸⁷Sr/⁸⁶Sr_{□n}·□⁸⁷Sr/⁸⁸Sr_{□n}]₁□□v₁₄·v_{23□}/□v₁₃·v_{24□}
[□⁸⁷Sr/⁸⁶Sr_{□n}·□⁸⁷Sr/⁸⁸Sr_{□n}]₂□□v₂₃·v_{32□}/□v₂₂·v_{33□}
and one or both of the following two kinds formulas:
for undiluted analysis:
(⁸⁸Sr/⁸⁶Sr)n=8.3752□
for diluted analysis:
[S₁−□⁸⁴Sr/⁸⁶Sr_{□n}]/[□⁸⁴Sr/⁸⁶Sr_{□n}−N₁]□[S₂−□⁸⁸Sr/⁸⁶Sr_{□n}]/[□⁸⁸Sr/⁸⁶Sr_{□n}−N₂]
and
Nᵢ□□⁸⁷Sr/⁸⁶Sr_{□n}+[□⁸⁷Sr/⁸⁶Sr_{□n}−Sᵢ]·□N₁−□⁸⁴Sr/⁸⁶Sr_{□n}□/□□⁸⁴Sr/⁸⁶Sr_{□n}−S₁□□
where (⁸⁴Sr/⁸⁶Sr_{)n,} □⁸⁷Sr/⁸⁶Sr_{□n} and (⁸⁸Sr/⁸⁶Sr)ₙ are normalized diluted ratios, S1,Si and S2 denote as the known isotope ratios ⁸⁴Sr/⁸⁶Sr□ ⁸⁷sr/⁸⁶Sr and ⁸⁸Sr/⁸⁶Sr of the spike respectively, N1 and N2 denotes naturally occurring isotope ratios ⁸⁴Sr/⁸⁶Sr and ⁸⁸Sr/⁸⁶Sr of strontium and Ni denote the isotope ratio□⁸⁷Sr/⁸⁶Sr_{□n} of the sample to which the contribution of spike has been corrected □ solutions of the ratio□⁸⁷Sr/⁸⁶Sr_{□n} will be
[□⁸⁷Sr/⁸⁶Sr_{□n}]₁□\sqrt{(V₁₄·V₂₃)·K/(V₁₃·V₂₄)}
[□⁸⁷Sr/⁸⁶Sr_{□n}]₂□\sqrt{(V₂₃·V₃₂)·K/(V₂₂·V₃₃)}
where K is one of the solutions of □⁸⁸Sr/⁸⁶Sr_{□n} of the simultaneous equations, two values of □⁸⁷Sr/⁸⁶Sr_{□n} can be obtained in one cycle of scan.

2. Method as claimed in claim 1, wherein
after no less than five times measuring by virtual peak jumping, all of the output voltages of A1, A2, A3, A4 and A5 are read as vij (i=1, 2, . . . , 5, j=1, 2, . . . , 5) every time;
Solving simultaneous equations of the combination of the equations:
[□⁸⁴Sr/⁸⁶Sr_{□n}·□⁸⁸Sr/⁸⁶Sr_{□n}]₁□□v₁₁·v_{33□}/□v₁₃·v_{31□}□
[□⁸⁴Sr/⁸⁶Sr_{□n}·□⁸⁸Sr/⁸⁶Sr_{□n}]₂□□v₄₃·v_{15□}/□v₄₅·v_{13□}□
[□⁸⁷Sr/⁸⁶Sr_{□n}·□⁸⁷Sr/⁸⁸Sr_{□n}]₁□□v₁₄·v_{23□}/□v₁₃·v_{24□}□
[□⁸⁷Sr/⁸⁶Sr_{□n}·□⁸⁷Sr/⁸⁸Sr_{□n}]₂□□v₂₃·v_{32□}/□v₂₂·v_{33□}□
[□⁸⁷Sr/⁸⁶Sr_{□n}·□⁸⁷Sr/⁸⁸Sr_{□n}]₃□□v₃₂·v_{41□}/□v₃₁·v_{42□}□
[□⁸⁷Sr/⁸⁶Sr_{□n}·□⁸⁷Sr/⁸⁸Sr_{□n}]₄□□v₄₁·v_{55□}/□v₄₅·v_{51□}□
and one or both of the following two kinds formulas:
for undiluted analysis:
(⁸⁸Sr/⁸⁶Sr)_{n=8.37521,}
for diluted analysis:
[S₁−□⁸⁴Sr/⁸⁶Sr_{□n}]/[□⁸⁴Sr/⁸⁶Sr_{□n}−N₁]□[S₂−□⁸⁸Sr/⁸⁶Sr_{□n}]/[□⁸⁸Sr/⁸⁶Sr_{□n}−N₂]
and
Nᵢ□□⁸⁷Sr/⁸⁶Sr_{□n}+[□⁸⁷Sr/⁸⁶Sr_{□n}−Sᵢ]·□N₁−□⁸⁴Sr/⁸⁶Sr_{□n}□/□□⁸⁴Sr/⁸⁶Sr_{□n}−S₁□,
wherein (⁸⁴Sr/⁸⁶Sr_{) n,} □⁸⁷Sr/⁸⁶Sr_{□n} and (⁸⁸Sr/⁸⁶Sr₎ₙ are normalized dilution ratios ; S1,Si and S2 denote the isotope ratios ⁸⁴Sr/⁸⁶Sr□⁸⁷Sr/⁸⁶Sr and ⁸⁸Sr/⁸⁶Sr of the spike respectively; N1 and N2 denotes naturally occurring isotope ratios ⁸⁴Sr/⁸⁶Sr and ⁸⁸Sr/⁸⁶Sr of strontium and Ni denotes the isotope ratio □⁸⁷Sr/⁸⁶Sr_{□n} of the sample to which the contribution of spike has been corrected;
solutions of the ratio □⁸⁷Sr/⁸⁶Sr_{□n} will be:
[□⁸⁷Sr/⁸⁶Sr_{□n}]₁□\sqrt{(V₁₄·V₂₃)·K/(V₁₃·V₂₄)}
[□⁸⁷Sr/⁸⁶Sr_{□n}]₂□\sqrt{(V₂₃·V₃₂)·K/(V₂₂·V₃₃)}
[□⁸⁷Sr/⁸⁶Sr_{□n}]₃□\sqrt{(V₃₂·V₄₁)·K/(V₃₁·V₄₂)}
[□⁸⁷Sr/⁸⁶Sr_{□n}]₄□\sqrt{(V₄₁·V₅₅)·K/(V₅₁·V₄₅)}
where K is one of the solutions of□⁸⁸Sr/⁸⁶Sr_{□n} of the equations, four values of □⁸⁷Sr/⁸⁶Sr_{□n} can be obtained in one cycle of scan.

3. Method as claimed in claim 1, including the step of: the normalized mixed ratios □⁸⁴Sr/⁸⁶Sr_{□n} and □⁸⁸Sr/⁸⁶Sr_{□n} of the diluted sample can be obtained as solutions of the said simultaneous equations.

4. Method as claimed in claim 2, including the step of: the normalized mixed ratios □⁸⁴Sr/⁸⁶Sr_{□n} and □⁸⁸Sr/⁸⁶Sr_{58 n} of the diluted sample can be obtained as solutions of the said simultaneous equations.

5. Method as claimed in claim 1, wherein the said spectrometer includes amplifiers coupled to the multi-collector, the signals of voltages expressed as a two dimensions collectivity v1j, v2j , v3j are dynamically read via the amplifiers

6. Method as claimed in claim 2, wherein the said spectrometer includes amplifiers coupled to the multi-collector, the signals of voltages expressed as a two dimensions collectivity v1j, v2j , . . . , v5j are dynamically read via the amplifiers

7. Method as claimed in claim 5, wherein a controllable relay matrix is coupled between the said multi-collector and amplifiers to establish the open and close connection between the said multi-collector and amplifiers, so as to read the signal of voltage expressed as a spectrum matrix [vij] where i=1, 2, 3 and j=1, 2, . . . , 5 no less than three times in one cycle of scan.

8. Method as claimed in claim 6, wherein a controllable relay matrix is coupled between the said multi-collector and amplifiers to establish the open and close connection between the said multi-collector and amplifiers, so as to read the signal of voltage expressed as a spectrum matrix [vij] where i=1, 2, . . . , 5 and j=1, 2, . . . , 5 no less than five times in one cycle of scan.

9. Method as claimed in claim 1, wherein when the dilution degree changes to zero, □⁸⁴Sr/⁸⁶Sr_{□n}—>N1, the second term in the following formula will be zero and the formula:
Nᵢ□□⁸⁷Sr/⁸⁶Sr_{□n}+[□⁸⁷Sr/⁸⁶Sr_{□n}−Sᵢ]·□N₁−□⁸⁴Sr/⁸⁶Sr_{□n}□/□□⁸⁴Sr/⁸⁶Sr_{□n}−S₁□
should be still valid, this kind of zero spike degree analysis can be called “virtual dilution analysis of strontium”.
